(19) United States
(12) Patent Application Publication
(10) Pub. No.: US 2020/0046722 A1
(43) Pub. Date: Feb. 13, 2020

(54) METHOD AND COMPOSITION FOR TREATING EATING DISORDERS

(71) Applicant: India Globalization Capital, Inc., Bethesda, MD (US)

(72) Inventor: Ramachandra MUKUNDA, Potomac, MD (US)

(73) Assignee: India Globalization Capital, Inc., Bethesda, MD (US)

(21) Appl. No.: 16/488,912

(22) PCT Filed: Feb. 27, 2018
(86) PCT No.: PCT/US2018/019814

§ 371 Date: Aug. 26, 2019

Related U.S. Application Data
(60) Provisional application No. 62/600,691, filed on Feb. 28, 2017.

(51) Int. Cl. A61K 31/55; A61K 36/185; A61K 31/05; A61K 31/353; A61K 31/519; A61K 31/714

(52) U.S. Cl. CPC A61K 31/55; A61K 36/185; A61K 31/05; A61K 2236/37; A61K 31/519; A61K 31/714; A61K 31/353

(57) ABSTRACT

Method and composition for treating eating disorders in humans and veterinary animals by administering a composition including: (i) mirtazapine, (ii) a cannabis compound and (iii) a fat-soluble vitamin.

### CROSS-REFERENCE TO RELATED APPLICATIONS

The present application claims priority on prior U.S. Provisional Application Ser. No. 62/600,691, filed Feb. 28, 2017, which is hereby incorporated herein in its entirety by reference.

### FIELD AND BACKGROUND OF THE INVENTION

This invention relates to compositions and methods for treating multiple types of eating disorders including cachexia in humans and animals (mammals) using a combination of a cannabis compound such as a cannabinoid extracted from the cannabis plant, mirtazapine, and a fat-soluble vitamin to inhibit degradation of mirtazapine and the cannabis compound to increase the amount of bioavailable mirtazapine and cannabis compound.

About 1.3 million humans in the United States are affected each year by cachexia which is a weakness and wasting away of the body due to severe illness such as cancer, multiple sclerosis, Parkinson's disease, HIV/AIDS and other progressive illnesses. Cachexia is secondary to an underlying disease such as cancer or AIDS and is a positive risk factor for death. It is often seen at the end-stage of cancer. (Payne, et al. 2012; Rapini et al. 2007).

Cancer induced cachexia is responsible for about 20% of all cancer deaths. It physically weakens patients to the extent that response to standard treatments is poor. (Lainscak, et al. 2007; Bossola, et al. 2007).

Studies have shown that non-drug therapies such as nutritional counseling, psychotherapeutic interventions, and physical training can be an effective treatment for cachexia. Treatments involving a combination of nutrition, medication and non-drug treatment have been more effective than mono-therapy. These studies suggest that cannabinoids not be used to treat cachexia due to a lack of conclusive evidence of efficacy or safety. (European Palliative Care Research Collaborative. New European Guidelines:

Mirtazapine is sometimes prescribed as an appetite stimulant for cats or dogs that are experiencing anorexia and has been shown to garner weight gain, albeit with side effects such as elevated levels of serotonin, increased heart rate, tremors, hyperactivity, fever, and high blood pressure. Studies have shown that in comparison with placebo, cats ingested significantly more food and had a greater appetite when mirtazapine was administered. (Quimby, J. M et. al. Journal of Veterinary Pharmacology and Therapeutics 2011; Quimby, J. M. The Veterinary Journal 2013).

Mirtazapine is a tetracyclic antidepressant used for the treatment of moderate to severe depression. It is commonly classified as a noradrenergic and specific serotonergic antidepressant (NaSSA). While its primary use is the treatment of major depressive disorder and other mood disorders, it has been found useful in alleviating may other conditions including insomnia, nausea, itching, Post-traumatic stress disorder and low appetite. (Davis, M P et al 2002; Chiu, H W 2011)

The appetite-stimulating effect of Mirtazapine has been reported in humans with advanced cancer, cachexia, cystic fibrosis and other diseases. (Davis M. P. et al Expert Review of Anticancer Therapy 2002; Riechelmann, R. P., et. al. American Journal of Hospice and Palliative Medicine (2009); Chinuck, R. S. et. al. Journal of Human Nutrition and Dietetics, 2007)

The appetite-stimulating effect of cannabis has been reported in anecdotal cases. (Garter, 1999; Felder, et al. 1998; Mikuriya, et al. 1969;).

However, in 2006, a multicenter, Phase III, randomized, double-blind, placebo-controlled, clinical trial on patients with cancer related anorexia-cachexia syndrome, reported that there was no difference between a placebo, cannabis extracts, or Delta-9-tetrahydrocannabinol (THC), at the dosage administered, in patient's quality of life, or appetite. (Strasser F. et. al. Journal of Clinical Oncology, 2006).

### SUMMARY OF THE INVENTION

The invention provides a method for treating eating disorders in humans and veterinary animals which includes administering to a subject in need thereof a composition including (i) an effective amount of mirtazapine, (ii) a cannabis compound in a dosage amount sufficient to inhibit degradation of mirtazapine; and (iii) a fat-soluble vitamin in an amount effective to inhibit degradation of mirtazapine and cannabis compound thereby increasing the amount of bioavailable mirtazapine and cannabis compound to said subject.

Compositions of the invention for treating eating disorders in humans and veterinary animals include: (i) an effective amount of mirtazapine, (ii) a cannabis compound in a dosage amount sufficient to inhibit degradation of mirtazapine; and (iii) a fat-soluble vitamin in an amount effective to inhibit degradation of mirtazapine and cannabis compound thereby increasing the amount of bioavailable mirtazapine and cannabis compound to said subject.

A water-soluble vitamin like folic acid can be administered separately to reduce side effects or it can be included in the composition.

### DETAILED DESCRIPTION OF THE PREFERRED EMBODIMENT(S) OF THE INVENTION

Cannabis compounds can be synthetic (chemically synthesized) or extracted from cannabis plants such as sativa, indica, hemp or hybrid strains of sativa and indica. A preferred source of cannabidiol (CBD) is so-called organic CBD, which is extracted from cannabis and contains minor amounts of other cannabinoids such as THC.

The invention also provides a method for treating eating disorders including anorexia, and cachexia, in mammals by administering to a subject in need thereof a composition including: (i) an effective amount of mirtazapine; (ii) a cannabis compound such as CBD in a dosage amount sufficient to inhibit degradation of mirtazapine; and (iii) a fat-soluble vitamin, like vitamin E, in an amount effective to inhibit degradation of mirtazapine and the cannabis compound thereby increasing the amount of bioavailable mirtazapine and CBD to a patient.

The preferred maximum dose of mirtazapine is about 1 mg/day for each kg of patient body weight, the minimum dose of CBD is about 10 mg/day for each kg of patient body weight up to a maximum of about 300 mg/day and the dose for Vitamin E is 400 International Units (IU) to 1000 IU per dose depending on BMI. The invention allows for the use of low amounts of mirtazapine with greater amounts of cannabis compound, thus reducing the side effects of mirtazapine, for example in the ratio of about 1:10 up to about 1:50.

Mirtazapine is: 1,2,3,4,10,14b-hexahydro-2-methylpyrazino[2,1-α]pyrido[2,3-c]benzazepine. It is sold under many brand names including Remeron, Avanza, Mirtazon, and Zispin among others. The mirtazapine analog mianserin, sold under the brand names Tolvon and Lerivon, has similar pharmacological properties and can be used in place of mirtazapine. Mirtazapine is preferred.

Mirtazapine is a tetracyclic that in animal studies has been shown to be have noradrenergic serotonergic effects. It is referred to as having dual modes of action. Though no demonstrable serotonergic effects in humans has been shown with insufficient evidence to designate it as a dual-action drug. (Gillman P K, 2006). It acts by antagonizing the adrenergic α2-autorecpetors and α2-heteroreceptors as well as blocking 5-HT2 and 5-HT3 receptors. Therefore, it enhances the release of norepinephrine and 5-HT1A-mediated serotonergic transmission; blockade of these receptors may explain the increase in appetite. It also exhibits significant antagonism at H1-receptors. For our case: “Mirtazapine is extensively metabolized in the liver. The cytochrome (CYP) P450 and isoenzymes CYP1A2, CYP2D6 and CYP3A4 are mainly responsible for it metabolism”. (Anttila, S. A. K., and E. V. J. Leinonen CNS Drug Reviews 2001).

The combination of mirtazapine and cannabis is believed to work on two different pathways in controlling cachexia and eating disorders by increasing the desirability of food and increasing appetite. It is believed that using CBD in combination with a fat-soluble vitamin like vitamin E is believed to overcome the metabolic effect of CYP450 and increase the bioavailability for mirtazapine and CBD, thereby requiring lower doses. Bioavailability can be further enhanced by using time-release formulations for any of the components, especially mirtazapine.

The combination of lower dose of mirtazapine, cannabis compounds and vitamin E unexpectedly leads to: i) a reduction of side effects, such as dry mouth, somnolence, and constipation, otherwise present when mirtazapine is used alone; and ii) increased appetite over using THC, cannabidiol (CBD), cannabis Extract (CE), other cannabinoids, or synthetic forms of these compounds.

Suitable pharmaceutically acceptable cannabis compounds include cannabis extract, which includes phytocannabinoids such as tetrahydrocannabinol “THC” (9-Tetrahydrocannabinol (delta-9 THC), 8-tetrahydrocannabinol (Delta-8 THC) and 9-THC Acid), cannabidiol (CBD), other phytocannabinoids such as cannabinol (CBN), cannabichromene (CBC), cannabigerol (CBG) among others, terpenoids and flavonoids. Standardized cannabis extract (SCE) consists of mostly THC, CBD and CBN. Organic CBD consists of solvent extracted CBD from cannabis with lesser or trace amounts of other cannabinoids, terpenoids and flavonoids. Synthetic or pure CBD is free of THC and other compounds is a preferred cannabis compound.

THC and CBD can be extracted from a cannabis indica dominant strain using, for example, high pressure and carbon dioxide or ethanol as a solvent in a 1500-20 L subcritical/supercritical CO₂ system made by Apeks Super Critical Systems, 14381 Blamer Rd., Johnstown, Ohio, 43031.

Another source of CBD essentially free of THC is the CBD mixture obtained from hemp or by extracting hempseed oil. See Leizer et al, J. Nutraceuticals, Functional and Medical Foods, Vol. 2(4) 2000, The Haworth Press, Inc. Elixinol (D&G Health LLC) is a predominantly CBD product extracted from hempseed oil that contains trace amounts of THC.

The preferred blocking compound is a natural or synthetic fat-soluble vitamin normally stored in fatty tissue such as vitamins A, D, E and K and mixtures thereof.

Vitamin A is a fat-soluble group of unsaturated compounds that includes retinol, retinal, retinoid acid, beta-carotene and other provitamin A carotenoids.

Vitamin D is a fat-soluble secosteroid such as cholecalciferol and ergocalciferol.

Vitamin E is commonly gamma-tocopherol from corn or soybean oil, or alpha-tocopherol from wheat germ oil or sunflower and safflower oils. Vitamin E is preferred because it is less likely to cause hypervitaminosis E.

Vitamin K is synthesized by plants and is a family 2-methyl-1,4-naphthoquinone (3-) derivatives.

Natural or synthetic water-soluble vitamins can be used to reduce side effects and boost the immune system and include folic acid, folate, vitamin B9 and vitamin B12.

The preferred water-soluble vitamin is folic acid, which is the synthetic form of vitamin B also known as pteroylglutamic acid.

The preferred maximum dose of mirtazapine is about 1 mg/day for each kg of patient body weight. Because of the P450 blocking effect provided by the other compounds, the bioavailability of mirtazapine and CBD is increased, which allows the use of lesser amounts of mirtazapine with a concomitant lowering in undesirable side effects normally seen with mirtazapine. Thus, it is preferred to use mirtazapine dosages of about 10 to 90% less than the normal dosage when the drug is given alone.

The dosage of CBD to be used with mirtazapine is from 10 mg/day/ per kg of patient body weight up to a maximum of about 300 mg/day

The dosage of fat-soluble vitamin, especially Vitamin E is about 400 International Units (IU) to 1000 IU per day depending on BMI.

A water-soluble vitamin, especially folic acid, can be administered separately at from about 1 to about 20 mg/day or compounded with the other components in a dosage amount of about 0.5 to about 1.0 mg/kg of patient weight.

Animals, especially dogs and cats, can be treated according to the invention. Dosage amounts and serum levels of drug are the same as disclosed above for human patients.

Those skilled in the art will recognize, or be able to ascertain many equivalents to the specific embodiments of the invention described herein.

While this invention has been described as having preferred sequences, ranges, ratios, steps, order of steps, materials, structures, symbols, indica, sativa, hemp, graphics, color scheme(s), shapes, configurations, features, components, or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the claims appended hereto or presented later. The invention, therefore, is not limited to the preferred embodiment(s) shown/described herein.

### REFERENCES

Payne, C., P. J. Wiffen, and S. Martin. “Interventions for fatigue and weight loss in adults with advanced progressive illness.” The Cochrane Library (2012).

Rapini, R. P., Bolognia, J. L., Jorizzo, J. L. Dermatology: 2-Volume Set. St. Louis: Mosby (2007): 1169.

Lainscak, M. Podbregar, M., Anker, S. D. “How does cachexia influence survival in cancer, heart failure and other chronic diseases?” Current Opinion in Supportive and Palliative Care 1.4 (2007): 299-305.

Bossola, M. Pacelli, F., Doglietto, G. B. “Novel treatments for cancer cachexia”. Expert Opinion Investigating Drugs 16.8 (2007): 1241-1253.

Suzuki, H, A. Asakawa, H. Amitani, N. Nakamura and A. Inui. “Cancer cachexia-pathophysiology and management” Journal of Gastroenterology 48.5 (2013): 574-594.

Ronga, I., F. Gallucci, F. Riccardi, G. Uomo. “Anorexia-cachexia syndrove in pancreatic cancer: recent advances and new pharmacological approach” 59.1 Advances in Medical Sciences (2014): 1-6.

Gorter, R. W. “Cancer Cachexia and Cannabinoids” Research in Complementary Medicine (1999): 021-022.

Felder, C. C. and M. Glass. “Cannabinoid receptors and their endogenous agonists” Annual Review of Pharmacology and Toxicology 38.1 (1998): 179-200.

Mikuriya, T. H. “Marijuana in medicine: past, present and future.” California Medicine 110.1 (1969):34.

Aquino, G. and D. Geffen. “Medical Marijuana: A Legitimate Appetite Stimulant?” Nutrition Bytes 10.1 (2005): 1-5.

Kirkham, T. C. and C. M. Williams. “Endogenous cannabinoids and appetite” Nutrition Research Reviews 14 (2001): 65-86.

Anttila, S A; Leinonen, E V (2001). “A review of the pharmacological and clinical profile of mirtazapine”. CNS Drug Reviews 7 (3): 249-64.doi:10.11 /j.1527-3458.2001.tb00198.x.PMID 11607047.

Chiu, H W; Li, T C (2011). “Rapid weight gain during mirtazapine treatment”. The Journal of Neuropsychiatry and Clinical Neurosciences 23 (1):

E7.doi:10.1176/appi.neuropsych.23.1.E7.PMID 21304130.

Davis, M. P., E. Khawam, L Pozuelo and R. Lagman. “Management of symptoms associated with advanced cancer: olanzapine and mirtazapine” Expert Review of Anticancer Therapy 2.4 (2002): 365-376

Chinuck, R. S., H. Fortnum, and D. R. Baldwin. “Appetite stimulants in cystic fibrosis: a systematic review” Journal of Human Nutrition and Dietetics : the Official Journal of the British Dietetic Association 20.6 (2007): 526-37

Quimby, J. M., D. L. Gustafson, B. J. Samber and K. F. Lunn. “Studies on the pharmacokinetics and pharmacodynamics of mirtazapine in healthy young cats” Journal of Veterinary Pharmacology and Therapeutics 34.4 (2011): 388-396

Quimby, J. M., and K. F. Lunn. “Mirtazapine as an appetite stimulant and anti-emetic in cats with chronic kidney disease: A masked placebo-controlled crossover clinical trial” The Veterinary Journal 197.3 (2013): 651-655

Cahill, C. “Mirtazapine as an antiemetic” Veterinary Forum (2006): 34-36

Haney, M., C I. Hart, S. K. Vosburg, S. D. Corner, S. C. Reed, Z. D. Cooper and R. W. Foltin. “Effects of Baclofen and Mirtazapine on a Laboratory Model of Marijuana Withdrawal and Relapse” Psychopharmacology 211.2 (2010): 233-244

Strasser F, Luftner D, Possinger K, et al. Comparison of orally administered cannabis extract and delta-9-tetrahydrocannabinol in treating patients with cancer-related anorexia-cachexia syndrome: a multicenter, phase Ill, randomized, double-blind, placebo-controlled clinical trial from the Cannabis-In-Cachexia-Study-Group. J Clin Oncol. 2006; 24(21): 3394-3400.

Gillman P K. A systematic review of the serotonergic effects of mirtazapine in humans: implications for its dual action status. Hum Psychopharmacol: March 2006: 117-125

Toshio Chow, Toyoko Hiroi, et. al. Isoform-selective metabolism of Mianserin by Cytochrome P-450 2D: The American Society for Pharmacology and Experimental Therapeutics Volume 27 No. 10: 1999

1. Method for treating eating disorders in humans and veterinary animals comprising administering to a subject in need thereof a composition comprising (i) an effective amount of mirtazapine, (ii) a cannabis compound in a dosage amount sufficient to inhibit degradation of mirtazapine; and (iii) a fat-soluble vitamin in an amount effective to inhibit degradation of mirtazapine and cannabis compound thereby increasing the amount of bioavailable mirtazapine and cannabis compound to said subject.

2. Method of claim 1 wherein the cannabis compound is selected from the group consisting of cannabis extract (CE), standardized CE, cannabidiol (CBD), synthetic CBD, organic CBD, tetrahydrocannabinol (THC), synthetic THC and mixtures thereof.

3. Method of claim 1 wherein the cannabis compound comprises CBD and up to 50% THC.

4. Method of claim 1 wherein the cannabis compound is derived from cannabis sativa, indica, hemp or a hybrid of any of the foregoing.

5. Method of claim 4 wherein the cannabis compound is extracted using heated gas or super critical or sub critical carbon dioxide.

6. Method of claim 1 wherein the fat-soluble vitamin is selected from the group consisting of vitamin A, vitamin D, vitamin E, vitamin K and D, E or K or mixtures of the foregoing.

7. Method of claim 1 wherein the composition includes a water-soluble vitamin.

8. Method of claim 7 wherein the water-soluble vitamin is selected from the group consisting of folic acid, folate, vitamin B9 and vitamin B12 and mixtures of the foregoing.

9. Method of claim 1 wherein the maximum dosage of mirtazapine is about 0.1 mg/day for each kg of patient body weight.

10. Method of claim 1 wherein the dosage of cannabis compound is from about 10 mg/day to about 300 mg/day.

11. Method of claim 1 wherein the daily dosage amount of fat-soluble vitamin is about 400-1000 IU/kg of patient body weight.

12. Method of claim 7 wherein the water-soluble vitamin is administered separately in amounts of about 1-20 mg/day.

13. Method of claim 1 wherein the daily dosage of water-soluble vitamin is about 0.5 mg/kg to about 1.0 mg/kg of patient body weight.

14. Method of claim 1 wherein the dosage of mirtazapine to cannabis compound is in a ratio of about 1:50 up to about 1:1500.

15. Method for treating eating disorders in humans and veterinary animals comprising administering to a subject in need thereof a composition comprising (i) an effective amount of mirtazapine, (ii) a cannabis compound selected from the group consisting of CBD and a mixture of CB and up to 50% THC in a dosage amount of mirtazapine to cannabis compound in a ratio of about 1:50 up to about 1:1500; (iii) a fat-soluble compound selected from the group consisting of vitamins A, D, E, K and mixtures of the foregoing in an amount effective to inhibit degradation of mirtazapine and cannabis; and (iv) a water-soluble vitamin selected from the group consisting of folic acid, folate, vitamin B9 and vitamin B12 and mixtures of the foregoing, thereby increasing the amount of bioavailable mirtazapine and cannabis compound to said subject.

16. Composition for treating eating disorders in humans and veterinary animals comprising: (i) an effective amount of mirtazapine, (ii) a cannabis compound in a dosage amount sufficient to inhibit degradation of mirtazapine; and (iii) a fat-soluble vitamin in an amount effective to inhibit degradation of mirtazapine and cannabis compound thereby increasing the amount of bioavailable mirtazapine and cannabis compound to said subject.

17. Composition of claim 16 wherein the cannabis compound is selected from the group consisting of CE, standardized CE, CBD, synthetic CBD, organic CBD, THC, synthetic THC and mixtures thereof.

18. Composition of claim 17 wherein the cannabis compound comprises CBD and up to 50% THC.

19. Composition of claim 16 wherein the cannabis compound is derived from cannabis sativa, indica, hemp or a hybrid of any of the foregoing.

20. Composition of claim 19 wherein the cannabis compound is extracted using heated gas or super critical or sub critical carbon dioxide.

21. Composition of claim 16 wherein the fat-soluble vitamin is selected from the group consisting of vitamin A, vitamin D, vitamin E, vitamin K and D, E or K or mixtures of the foregoing.

22. Composition of claim 16 wherein the composition includes a water-soluble vitamin.

23. Composition of claim 22 wherein the water-soluble vitamin is selected from the group consisting of folic acid, folate, vitamin B9 and vitamin B12 and mixtures of the foregoing.

24. Composition of claim 16 wherein the maximum dosage of mirtazapine is about 0.1 mg/day for each kg of patient body weight.

25. Composition of claim 16 wherein the dosage of cannabis compound is from about 10 mg/day to about 300 mg/day.

26. Composition of claim 16 wherein the daily dosage amount of fat-soluble vitamin is about 100-2000 IU/kg of patient body weight.

27. Composition of claim 22 wherein the water-soluble vitamin is administered separately in amounts of about 1-20 mg/day.

28. Composition of claim 22 wherein the daily dosage of water-soluble vitamin is about 0.5 mg/kg to about 1.0 mg/kg of patient body weight.

29. Composition of claim 16 wherein the dosage of mirtazapine to cannabis compound is in a ratio of about 1:50 up to about 1:1500.

30. Composition of claim 16 wherein the for treating eating disorders in humans and veterinary animals comprising: (i) an effective amount of mirtazapine, (ii) a cannabis compound is selected from the group consisting of CBD and a mixture of CB and up to 50% THC in a dosage amount of mirtazapine to cannabis compound in a ratio of about 1:50 up to about 1:1500; the fat-soluble compound is selected from the group consisting of vitamins A, D, E, K and mixtures of the foregoing in an amount effective to inhibit degradation of mirtazapine and cannabis compound; and the composition further comprises a water-soluble vitamin selected from the group consisting of folic acid, folate, vitamin B9 and vitamin B12 and mixtures of the foregoing, thereby increasing the amount of bioavailable mirtazapine and cannabis compound to said subject.
